(19) United States
(12) Patent Application Publication
(10) Pub. No.: US 2021/0228685 A1
(43) Pub. Date: Jul. 29, 2021

(54) IMPROVED WEIGHT LOSS THERAPY

(71) Applicant: BIOREG LLC, Rockwall, TX (US)

(72) Inventors: Puneeth IYENGAR, Coppell, TX (US); Rodney Elwood INFANTE, Rockwall, TX (US)

(21) Appl. No.: 17/262,579

(22) PCT Filed: Jul. 24, 2019
(86) PCT No.: PCT/US2019/043214

§ 371 Date: Jan. 22, 2021

Related U.S. Application Data
(60) Provisional application No. 62/702,685, filed on Jul. 24, 2018.

(51) Int. Cl. A61K 38/20; A61P 3/04; A61K 45/06; A61K 39/395; A61K 38/22

(52) U.S. Cl. CPC A61K 38/2093; A61P 3/04; A61K 38/2264; A61K 39/3955; A61K 45/06

(57) ABSTRACT

A pharmaceutical composition is provided comprising a first agent in an amount effective for causing both a peripheral effect and a central effect on weight loss, a second agent in an amount effective for causing at least a central effect on weight loss, and one or more pharmaceutically acceptable excipients, wherein the first agent and the second agent are different. Also provided are a method of causing weight loss and a method of preventing or treating cachexia syndrome.

This patent application is based on and claims priority to U.S. Provisional Application Ser. No. 62/702,685, filed Jul. 24, 2018, the entire contents of which are herein incorporated by reference.

### TECHNICAL FIELD

The presently disclosed subject matter relates to pharmaceutical compositions for causing weight loss, methods of causing weight loss, and methods of preventing or treating cachexia syndrome.

### BACKGROUND

Cachexia (CX) syndrome consists of loss of adipose and muscle mass, often despite little change in caloric intake (1). This paradox of weight loss in the setting of normophagia has confounded researchers and clinicians. CX occurs in chronic inflammatory states including infection, rheumatologic conditions, and heart failure and in ˜50% of cancer patients (2, 3). Most preclinical and clinical trials to reverse CX, including targeting of the immune system (anti-TNFα and anti-IL-6), appetite stimulation (agonists of ghrelin receptor and cannabinoid receptor), and muscle regeneration (androgen receptor against and myostatin), have failed (4). Previous efforts to suppress CX have focused on muscle atrophy rather than changes in adipose tissue (5). However, enhanced lipolysis has also been identified in cachectic cancer patients (6-8). Blocking lipolysis through the genetic ablation of adipose triglyceride lipase (ATGL) in mice administered cancer cells that induce CX had complete inhibition of adipose tissue loss compared with littermate controls (8). Partial blocking of adipose tissue loss was observed in mice with genetic ablation of hormone sensitive lipase (HSL).

Obesity leads to greater mortality and is a risk factor for other diseases, including diabetes and cancer (9, 10). Similar to CX, obesity also lacks sustainable treatment options. Empirically, obese patients with CX frequently have improvement in insulin resistance, joint pain, sleep apnea, and nonalcoholic fatty liver disease as they lose weight. Therefore, identification of factors and mechanisms that underlie CX may lead to new treatments for CX patients and may also provide insights for treatment of obesity and its related complications.

Leukemia inhibitory factor (LIF) is a member of the IL-6 cytokine family, which includes cardiotrophin-1, oncostatin M, and ciliary neurotrophic factor (CNTF), among others (11). LIF interacts through its cognate receptor on cell surfaces, LIFR-α, which forms a heterodimer with its coreceptor subunit gp130 to activate the JAK/STAT pathway (12). LIF regulates growth and differentiation of a wide variety of cell types and is involved in processes as disparate as inflammation, neural development, hematopoiesis, embryogenesis, and fertilization (13-15).

The earliest studies evaluating LIF in animals were intended to assess its effect on hematopoiesis. Interestingly, an unexpected off-target effect of LIF was a decrease in body weight (16). Intracerebroventricular injections of LIF adenovirus into mice also decreased body weight, with modest changes in food intake (17). Recently, LIF has been linked to muscle wasting in cancer CX (18). CNTF, another member of the IL-6 family, also decreased food intake and subsequent body weight in rodents (19, 20). CNTF was evaluated in a large phase 3 randomized trial in the context of amyotrophic lateral sclerosis as a CNS promoter of neuronal regeneration and function. An unexpected off-target effect of CNTF in this trial was significant weight loss (21, 22), further implicating this family in CX.

### SUMMARY

One aspect of the present disclosure relates to a therapeutic composition comprising a first agent in an amount effective for causing both a peripheral effect and a central effect on weight loss, a second agent in an amount effective for causing at least a central effect on weight loss, and one or more pharmaceutically acceptable excipients. The first agent and the second agent may be different. The first agent may include at least one member of the IL-6 cytokine family, such as leukemia inhibitory factor (LIF) and/or ciliary neurotropic factor (CNTF). Preferably, the first agent comprises leukemia inhibitory factor. The second agent in the composition may include leptin.

Another aspect of the present disclosure relates to a method of causing weight loss comprising administering to a subject a first agent in an amount effective for causing both a peripheral effect and a central effect on weight loss and a second agent in an amount effective for causing at least a central effect on weight loss. The first agent and the second agent may be different. The subject may suffer from at least one disease or condition selected from the group consisting of obesity, metabolic syndrome, nonalcoholic fatty liver disease, and diabetes mellitus. The first agent may include at least one member of the IL-6 cytokine family, such as leukemia inhibitory factor (LIF) and/or ciliary neurotropic factor (CNTF). Preferably, the first agent in the combination comprises leukemia inhibitory factor. The second agent in the combination may include leptin. The effective amount of leptin administered to the subject may be an amount that is sufficient to counteract a decrease in natural leptin secretion associated with increased levels of LIF in the subject. The method of causing weight loss may involve administration of the first agent and the second agent over a prolonged period of time. For example, each of the first agent and the second agent may be administered to the subject over a period of at least a week, over a period of at least a month, over a period of at least four months, or over a period of at least one year. The route of administration is not particularly limited. For example, either or both of the first agent and the second agent may be administered to the subject by at least one route selected from the group consisting of oral, transmucosal, topical, transdermal, intradermal, subcutaneous, inhalational, intrabronchial, pulmonary, intravenous, intraduodenal, intramuscular and intragastrical. Preferably, at least one of the first agent and the second agent is administered to the subject intravenously. Preferably, each of the first agent and the second agent is administered intravenously.

Another aspect of the present disclosure relates to a method of causing weight loss comprising administering to a subject peripherally a composition comprising an effective amount of an agent that has both a peripheral effect and a central effect on weight loss. The subject may suffer from at least one disease or condition selected from the group consisting of obesity, metabolic syndrome, nonalcoholic fatty liver disease, and diabetes mellitus. The peripheral effect on weight loss may include lipolysis. The lipolysis may occur in adipocytes. The composition administered for causing weight loss may include at least one member of the IL-6 cytokine family, such as leukemia inhibitory factor (LIF) and/or ciliary neurotropic factor (CNTF). Preferably, the composition comprises leukemia inhibitory factor. The composition may be administered to the subject enterally or parenterally. For example, the composition may be administered by at least one route selected from the group consisting of oral, transmucosal, topical, transdermal, intradermal, subcutaneous, inhalational, intrabronchial, pulmonary, intravenous, intraduodenal, intramuscular and intragastrical. Preferably, the composition is administered to the subject intravenously. According to this aspect, the method of causing weight loss may further comprise administering to the subject an effective amount of an additional agent that has a central effect on weight loss. Preferably, the additional agent comprises leptin.

Another aspect of the present disclosure relates to a method of preventing or treating cachexia syndrome comprising administering to a subject in need thereof an effective amount of an agent that inhibits adipose loss. Examples of the agent include compounds, molecules or substances that reduce gene expression level of leukemia inhibitory factor, reduce LIF secretion, or block leukemia inhibitory factor signaling. For example, the agent may be an antibody, e.g., a monoclonal antibody, against leukemia inhibitory factor.

### BRIEF DESCRIPTION OF THE DRAWINGS

FIG. 1 shows that recombinant LIF induces adipocyte lipolysis through ATGL using its canonical signaling pathway. FIG. 1A shows gel-filtration chromatography and SDS/PAGE of recombinant proteins. FIG. 1B-D show adipocyte lipolysis by the recombinant proteins. FIG. 1E shows Gp130 neutralization of LIF-induced adipocyte lipolysis.

FIG. 2A-E shows LIF treatment of Balb/c wild-type mice to demonstrate that LIF induces adipose tissue and body weight loss without persistent change in food intake in Balb/c mice.

FIG. 3 shows that LIF's central anorexic effect is reversed by decreased leptin signaling. FIG. 3A-C shows leptin levels relative to fat mass and food intake in recombinant Leukemia Inhibitory Factor (rLIF)-treated C57BL/6 mice. FIG. 3D-E shows hypothalamic STAT3 phosphorylation that is a consequence of continued LIF treatment with a leptin reduction, permitting the system to attempt to bring food intake back towards baseline.

FIG. 4 shows that LIF's central effect persists with co-administration of leptin. FIG. 4A-C demonstrate combination treatment of C57BL/6 mice with recombinant Leukemia Inhibitory Factor (rLIF) and leptin on food intake, fat loss, and body weight loss.

FIG. 5 shows that LIF induces a persistent decrease in body weight, adipose mass, and food intake in a genetic model of obesity, the ob/ob mice. FIG. 5A-C show recombinant Leukemia Inhibitory Factor (rLIF) treatment of C57BL/6 ob/ob mice. FIG. 5D shows Hypothalamic STAT3 phosphorylation of ob/ob mice.

FIG. 6 shows that LIF induces central and non-central effects in ob/ob mice. FIG. 6A-C show recombinant Leukemia Inhibitory Factor (rLIF) treatment of C57BL/6 ob/ob mice with pair fed controls.

FIG. 7 shows that LIF induces a persistent decrease in body weight, adipose mass, and food intake in another genetic model of obesity, the db/db mice. FIG. 7A-C show recombinant Leukemia Inhibitory Factor (rLIF) treatment of C57BL/6 db/db mice.

FIG. 8 shows that recombinant LIF (rLIF) acts on mouse models of diet-induced obesity to reduce weight, fat mass, and food intake. The rLIF (red) caused a significant decrease in fat mass FIG. 8A and body weight FIG. 8B when administered to mice compared to mice receiving only vehicle (black). Furthermore, the rLIF reduced food intake in the animals FIG. 8C.

FIG. 9 illustrates a working model of LIF-induced adipose tissue loss and leptin's response in cachexia. FIG. 9A Normal Homeostasis: In normal homeostasis, leptin levels that are proportional to adipose mass are reached to maintain baseline hypothalamic phosphorylated STAT3 resulting in normophagia. FIG. 9B Acute cachexia: Cachexia factors work peripherally on adipose tissue and centrally with leptin on the hypothalamus to increase phosphorylated STAT3 resulting in hypophagia. FIG. 9C Chronic cachexia: As body weight and adipose mass decrease in the setting of cachexia factors, there is a corresponding suppression of leptin secretion into serum decreasing the extent of its hypothalamic signaling. Overall, the increase in cachexia factor signaling and the decrease in leptin signaling of the hypothalamus have a net effect of returning phosphorylated STAT3 back to baseline levels, normalizing food intake.

### DETAILED DESCRIPTION OF THE INVENTION

This invention is concerned with promotion or prevention of weight loss in subjects in need of modification of their status with regard to body mass. Such patients may suffer from or be susceptible to conditions such as cachexia (CX), obesity, metabolic syndrome, nonalcoholic fatty liver disease, or diabetes mellitus. The agents used in therapy according to this invention are agents whose effects may be central or peripheral as described herein. Central effects as contemplated by this invention include any effect on the central nervous system that could influence behavior that ultimately results in a change in weight and fat mass among other metabolic parameters. This includes, but is not limited to, actions on any part of the brain or spinal cord (i.e. hypothalamus, hippocampus, etc) affecting feeding patterns, appetite, energy expenditure, etc. Peripheral effects as contemplated by this invention include any effect on non-central tissues, including but not limited to adipocytes/adipose, hepatocytes/liver, muscle, immune cells, etc. that contribute to changes ultimately leading to changes in body weight and fat mass among other metabolic parameters.

To identify factors that mediate CX, the present inventors developed an in vitro lipolysis assay to screen for proteins that are involved in cancer CX-associated adipose tissue loss. Using a cancer cell line that causes CX in vivo, the present inventors biochemically purified secretory factors that enhance lipolytic activity in adipocytes and identified leukemia inhibitory factor (LIF).

The present inventors identified LIF as a factor secreted from cancer cells that induced lipolysis. Immunodepletion of LIF from partially purified medium removed lipolysis activity. Recombinant LIF (rLIF) added to cultured adipocytes confirmed that LIF induced lipolysis. LIF-induced lipolysis was dependent on its receptor, LIFR-α, and coreceptor, gp130, which activated the STAT pathways¹ and, ultimately, adipose triglyceride lipase (ATGL). rLIF injected into WT and genetically obese mouse models as well as high fat diet-induced obesity models resulted in significant loss of adipose tissue. Interestingly, WT mice with an intact leptin axis chronically administered rLIF showed a decrease in body weight and adipose tissue, with little change in overall food intake. However, these mice demonstrated an early transient hypophagic state that returned to normophagia as leptin levels decreased. Persistent rLIF-induced hypophagia could be maintained in these mice by coadministration of recombinant leptin (rleptin), resulting in further body weight and adipose loss. Administration of rLIF to 2 hyperphagic murine models, leptin deficient (ob/ob) and leptin signaling-deficient (db/db) mice, showed a persistent decrease in food intake. Importantly, rLIF administration to ob/ob mice resulted in loss of fat mass and body weight compared with PBS controls that were pair fed, demonstrating that LIF has an effect independent of changes in food intake and leptin levels. These studies suggest that LIF has both a direct peripheral contribution (50%-60%) and an independent central contribution (40%-50%) promoting transient hypophagia, which leads to adipose tissue loss followed by leptin counterregulation, ultimately providing an explanation for normal food intake in CX. ¹ Moris R, Kernhaw N J, Babon J J. The molecular details of cytokine signaling via the JAK/STAT pathway Protein Science 2018 December; 27(12):1984-2009. doi: 10.1002/pro 3519 Review PMID: 30267440.

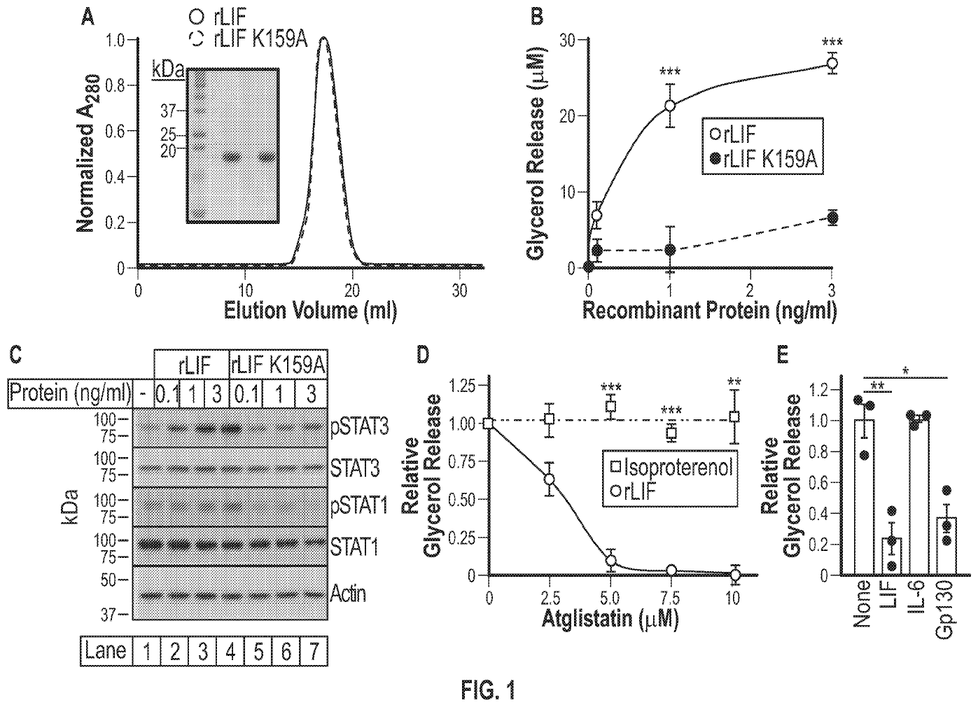

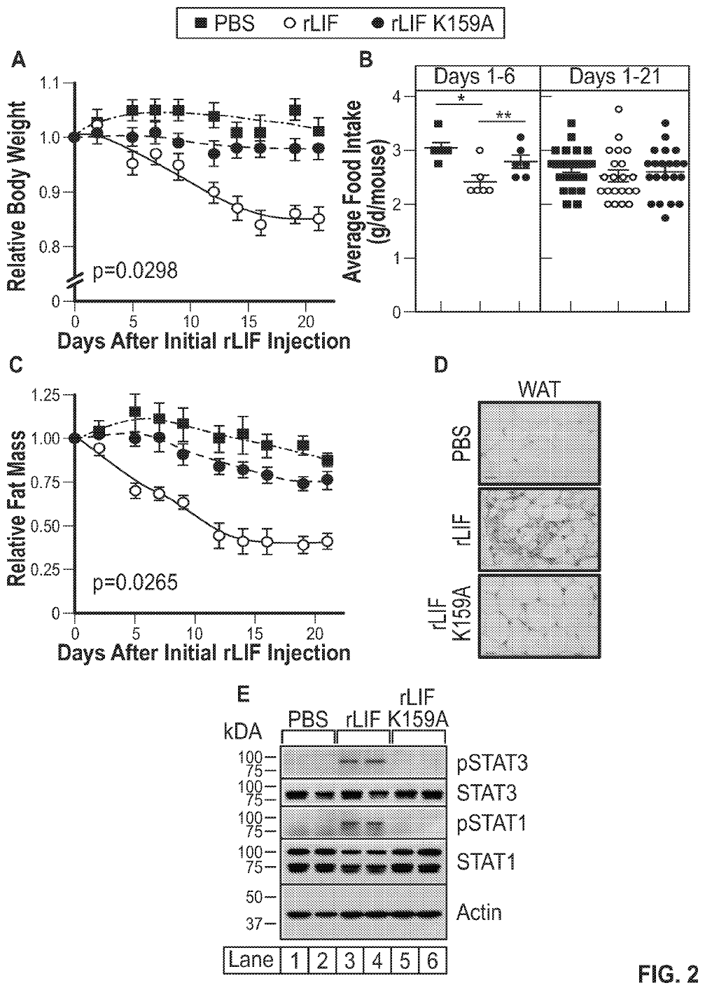

Originally, using an unbiased biochemical screen of secreted factors from a cell line that induces CX in vivo, the inventors identified LIF as a protein that induced lipolysis in adipocytes. LIF is most highly expressed by cancers that promote CX in humans—lung, renal cell, and gastrointestinal. Increased circulating levels of LIF were associated with multiple cancer CX mouse models displaying adipose loss. LIF was secreted from C26c20 cancer cells and induced lipolysis in adipocytes. LIF-induced lipolysis signaled through the coreceptor gp130 and resulted in STAT1 and STAT3 phosphorylation, as reported in other tissues. Unlike isoproterenol-induced lipolysis, which relies on HSL function, LIF-mediated lipolysis was dependent on ATGL activity (FIG. 1D). This finding is consistent with data from Das and colleagues (8), who showed adipose deletion of ATGL-protected mice from cancer-induced CX. The inventors subsequently showed that WT mice administered recombinant LIF (rLIF) that they had produced demonstrated adipose (FIG. 2C) and body weight loss (FIG. 2B), consistent with a CX phenotype.

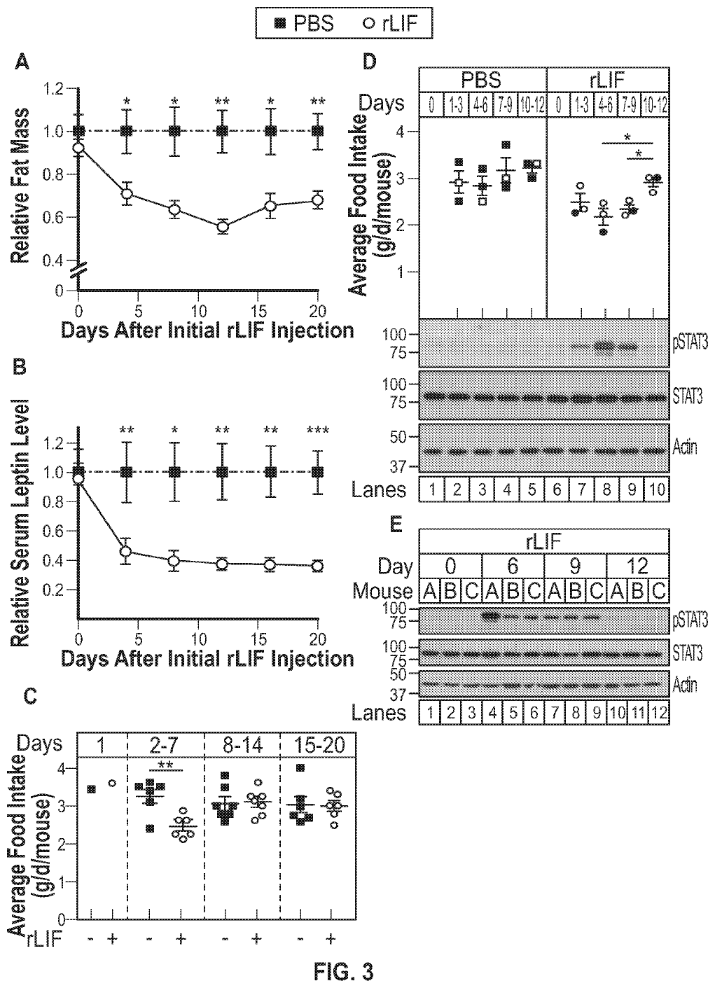

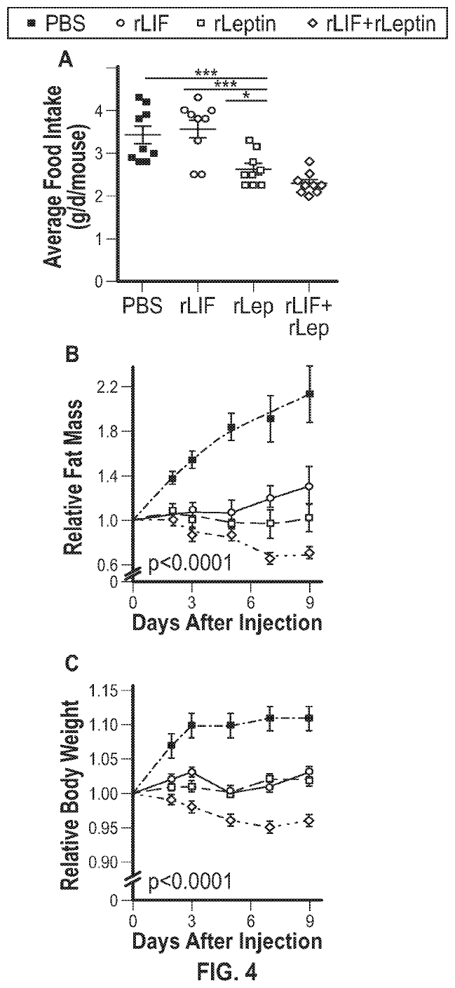

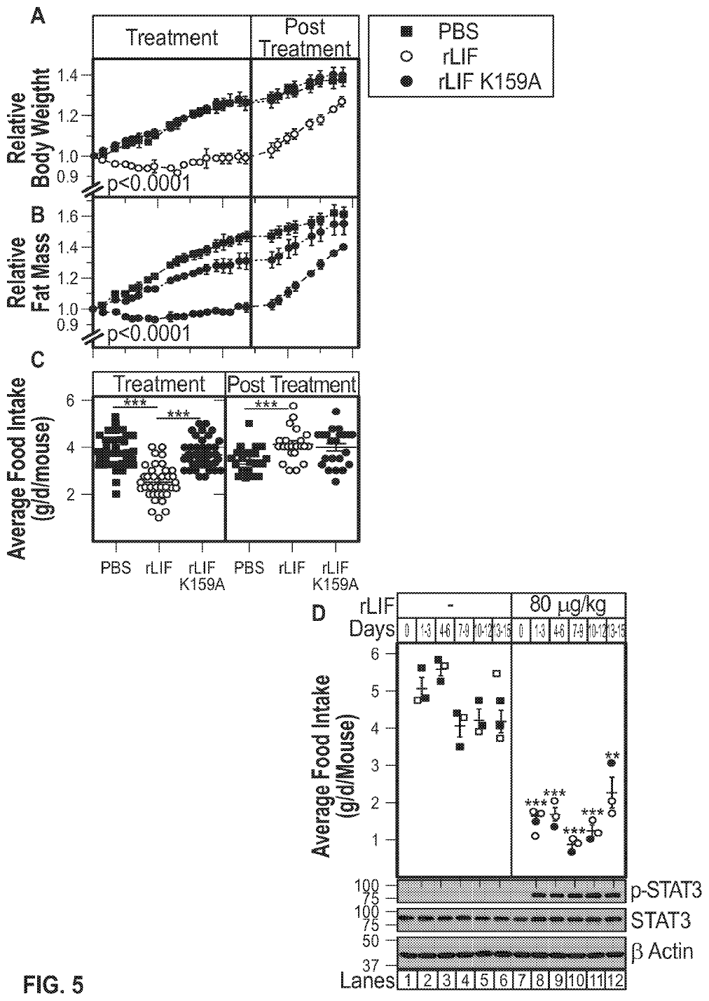

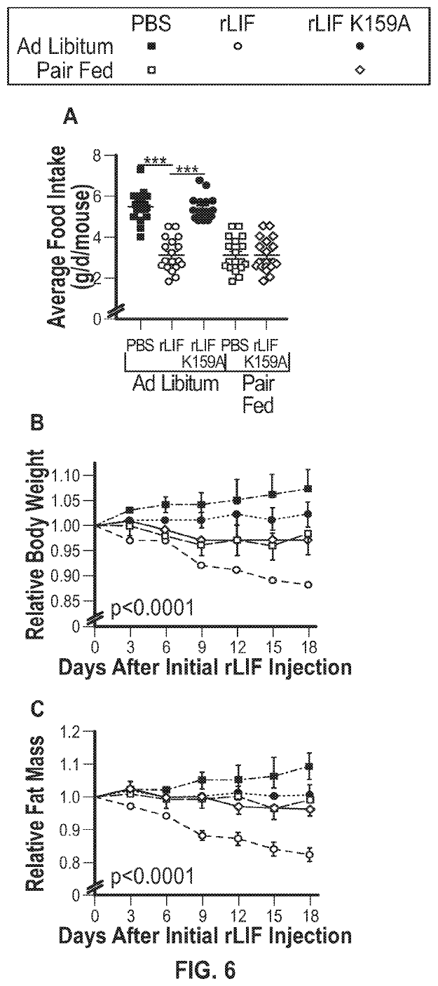

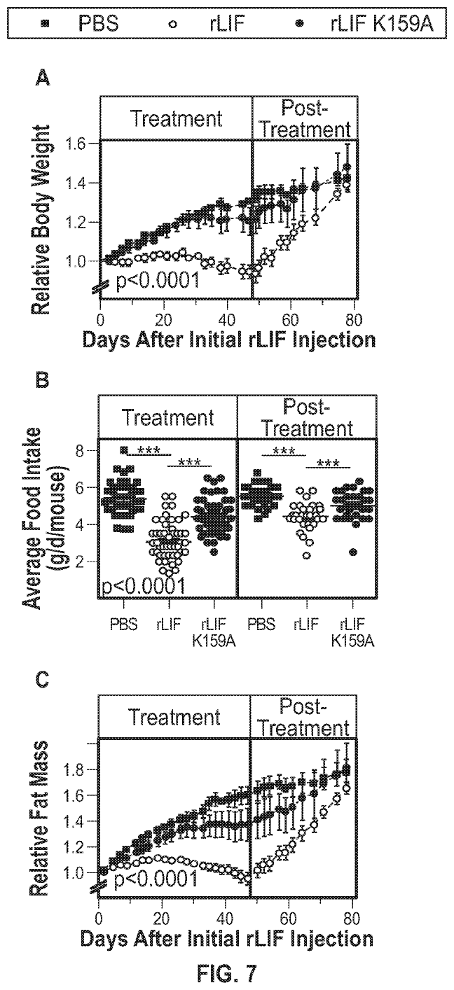

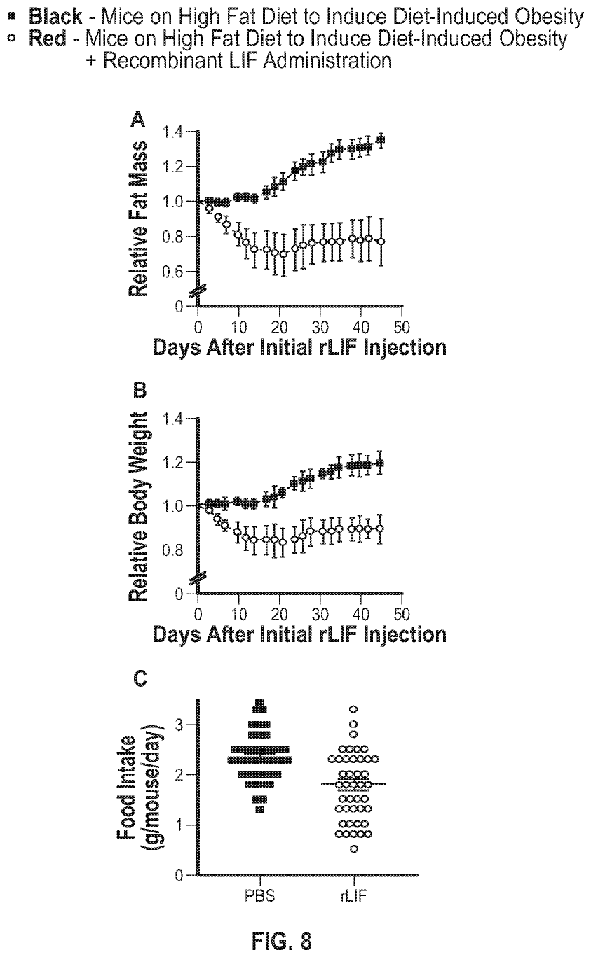

Although this in vitro assay confirmed that LIF induced lipolysis, the inventors identified additional functions for LIF in vivo. I.P. administration of rLIF to mice led to adipose tissue and body weight loss through both peripheral and central effects. LIF's central effect in WT mice manifested as transient hypophagia (FIG. 2B), possibly mediated through direct phosphorylation of STAT3 in the hypothalamus (FIG. 2E). LIF and leptin both can activate STAT pathways and signal through POMC neurons in the appetite center of the hypothalamus (30-32). Interestingly, LIF-induced body weight and adipose loss was associated with a transient reduction in food intake that paralleled a decrease in leptin (FIG. 3A-C). The reduction in leptin was associated with a decrease in STAT phosphorylation back to baseline levels and a stimulation in appetite-restoring food intake to pretreatment levels (FIG. 3D-E). This return of hypothalamic STAT phosphorylation and food intake back to baseline was not observed in the leptin-deficient ob/ob rLIF-treated mice (FIG. 5D). To confirm leptin was responsible for the change in food intake limiting rLIF-induced weight loss, the inventorsA coadministered rleptin and rLIF to the WT mouse model (FIG. 4) and B administered rLIF to leptin-deficient ob/ob mice (FIG. 5). Coadministering reptin to rLIF-chronically treated WT mice allowed rLIF's central effect of hypophagia to persist, resulting in further body weight and adipose loss (FIG. 4). Unlike WT mice, rLIF-administered ob/ob mice demonstrated a persistent decrease in food intake, which was also associated with a persistent decrease in body weight and adipose mass (FIG. 5). Leptin and LIF have similar long-chain helical structures (33) and both induce STAT activation. To determine if LIF signals through the leptin receptor, the inventors administered rLIF to leptin receptor-deficient db/db mice (FIG. 7). Similar to ob/ob mice, db/db mice also demonstrated a persistent decrease in food intake with an associated decrease in body weight and adipose mass, suggesting that LIF's signaling of the hypothalamus is independent of the leptin receptor. To determine if LIF-induced weight loss was solely due to decreased food intake, the inventors performed pair-feeding studies in rLIF-treated ob/ob mice (FIG. 6). The inventors found that rLIF administration decreased body weight and fat mass more than controls, suggesting that half of rLIF's contributions to adipose loss is secondary to its central effect of hypophagia (FIG. 6B) black circles, closed vs. open) and the other half is due to a peripheral effect (FIG. 6B), black open circles vs. red closed circles). Adipocyte-specific LIFR-α-null mice and POMC neuron-specific LIFR-α-null mice may be generated to quantify the relevant central versus peripheral contributions of rLIF on body weight and adipose loss. Finally, the inventors verified that LIF could also cause fat loss and body weight loss in a high fat diet-induced model of obesity (FIG. 8) with a concomitant reduction in food intake. Of note, the drop in fat loss plateaued, suggesting that leptin co-administration would have resulted in greater loss of fat mass and body weight.

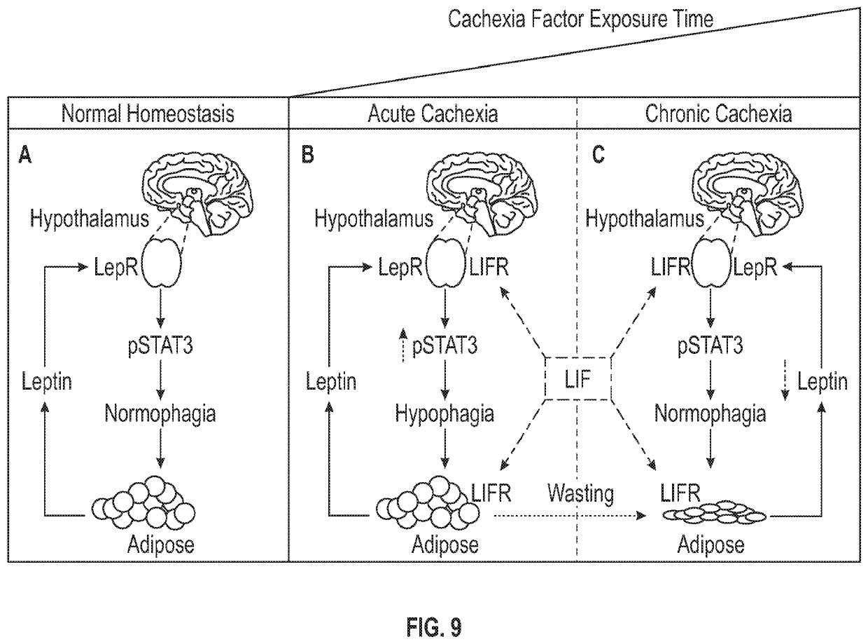

A working model that proposes how LIF induces adipose tissue loss in mice with an intact leptin axis is shown in FIG. 9. LIF has two major actions: it peripherally stimulates lipolysis in adipose tissue, and it centrally triggers early hypophagia by acting on the hypothalamus, likely through LIF receptor activation of STAT3 pathways. Both events occur during the early exposure to a CX factor(s), causing adipose tissue loss, which in turn leads to reduced leptin secretion over time. The chronic decrease in leptin results in a return of food intake approaching normophagia, owing to a decrease in hypothalamic STAT3 phosphorylation through the leptin receptor pathway. The increase in LIF receptor activation and the decrease in leptin receptor signaling consequently have a net effect of normalizing food intake in the setting of overall adipose tissue loss and decreased body weight. It is likely that LIF's noncentral effect on adipocyte lipolysis permits sustained differences in body weight despite normal food intake. This is consistent with data in FIG. 4, demonstrating that mice chronically treated with rLIF maintained their reduction in body weight and adipose mass, whereas those animals that stopped chronic rLIF treatment (PBS) regained their body weight and adipose mass in the setting of equivalent food intake.

The current studies provide insight into the clinical paradox of human CX patients who describe losing weight despite eating normally. The inventors have found that the extent of LIF's peripheral and central effects on body weight and adipose tissue loss is limited by leptin's counterregulation in WT animals with an intact leptin axis. In the clinical setting, patients with cancer CX continue to lose weight in the setting of perceived normophagia. One difference between the inventor's animal studies and clinical cancer CX patients is that mice were administered a constant amount of LIF throughout the entire experiment. Eventually, a steady state is reached that is determined by the balance of CX factor and leptin response. Patients have an ever-expanding cancer burden and immune response with increasing circulating levels of multiple CX factors over time, likely resulting in constant adjustment of leptin levels in an attempt to maintain normophagia. At the extreme end of the CX spectrum, in which leptin counterregulation has been exhausted, the phenotype can manifest as frank anorexia.

To block the murine model of cancer CX, a monoclonal antibody against LIF may be created, and the antibody may be expected to have neutralizing benefits in cachectic settings. However, the LIF antibody may only partially suppress cancer CX wasting, since multiple factors—including other IL-6 family members—are expressed by tumors/immune cells and offer similar wasting effects as LIF (20, 35, 36). The ability of chronic disease/immune conditions to stimulate a milieu of factors that affect whole body metabolism, food intake, and energy consumption may explain why single agents that target CX have likely failed to block this syndrome. The IL-6 family of ligands relies on their respective receptors and their common coreceptor, gp130, for signaling. Targeting this coreceptor or its downstream pathway may abrogate the central and peripheral effects of this class of cytokines on CX.

The present inventors found that LIF is a tumor-secreted molecule that promotes adipose tissue loss through two mechanisms: A) a peripheral effect that includes adipocyte lipolysis and B) a central effect that induces anorexia. The effect of LIF and IL-6 family members on sustained adipose wasting is counterbalanced by changes in leptin levels and signaling, limiting the extent of the central actions induced by these CX factors. This more detailed understanding of how LIF mediates lipolysis and anorexia presents new therapeutic opportunities to treat not only CX, but also obesity-related comorbidities.

### EXAMPLES

### Example 1

CX-Inducing C26c20 Cells Secrete Factors that Increase Adipocyte Lipolysis.

C26 represents an undifferentiated murine adenocarcinoma cell line created by chemical carcinogen induction in Balb/c mice followed by serial passage of resulting tumors in syngeneic mice. These tumor-bearing mice develop loss of fat and lean body mass (23). A clone of this cell line, C26c20, increased the amount of weight loss, adipose tissue loss, and muscle atrophy when injected s.c. into Balb/c mice (24). Considering that human colon adenocarcinoma is associated with CX (25), we reasoned that the C26c20 murine colon adenocarcinoma cell line is a potential model to identify secreted factors capable of inducing loss of fat mass.

To validate this cell line's potential to induce CX, we injected C26c20 cells or PBS in the right hind leg of syngeneic Balb/c mice. Chow-fed Balb/c mice (11-week-old males) were housed four mice per cage and injected s.c. in right flank with 100 μl PBS in the absence or presence of 1×10⁷ C26c20 cells at day 0. Animals were kept in a temperature-controlled facility with a 12-hour light/dark cycle and were fed normal chow diet and provided water ad libitum unless indicated. Approximately 100 g of standard chow diet (Envigo, Teklad global 16% protein irradiated rodent diet, catalog 2916) was placed in each cage. When food reached approximately 50 g per cage, it was replenished to approximately 100 g. Food was weighed at the same time daily and compared with the previous day's weight to calculate the 24-hour food intake per cage. Body weight was measured daily at the same time using a standard balance (digital Soenhlin scale). Adipose tissue mass and lean tissue mass were measured longitudinally using ECHO MRI (ECHO Medical Systems). Tumor volume was calculated by taking half of the product of the caliper (VWR) measurements of length, width and breath at the indicated time points. Body weight, food intake, and ECHO MRI measurements of fat mass and lean mass were measured daily at 9 a.m. Body weight, fat mass, and lean mass are relative to the average day 0 reference value for each respective cohort.

The average values for body weight at day 0 were 24.3 and 25.0 g for the PBS and C26c20-injected mice, respectively. The average values for fat mass at day 0 were 3.1 and 3.5 g for the PBS and C26c20 injected mice, respectively. The average values for lean mass at day 0 were 18.2 and 18.7 g for the PBS and C26c20 injected mice, respectively. These results were confirmed in three independent experiments. Each value represents mean±SEM of four mice. As the C26c20 tumor increased in size, both the body weight and adipose mass decreased compared with mice injected with PBS (data not shown). Lean mass and food intake showed no differences in C26c20-injected mice compared with PBS-injected mice (data not shown).

Whole blood was drawn from the tail vein, and serum was obtained by subjecting the whole blood to centrifugation at 960 g at 4° C. for 10 minutes. Supernatant was removed, followed by protein concentration quantification using a bicinchonianic acid kit (Pierce), and 5 μl from each condition was used in the leptin ELISA kit per manufacturer's instructions. The rest of the serum was stored at −80° C. for future blood analysis.

To test if the C26c20 cells had an intrinsic ability to induce adipocyte lipolysis, we developed an in vitro model. C26c20 cells were incubated for 20 hours in culture medium that did not contain phenol red or FBS. As a control, we used MC-38 cells, an undifferentiated murine colon adenocarcinoma line made similarly to the C26c20 line but one that does not induce the CX phenotype in allotransplant mouse models (26). Conditioned medium from the C26c20 and MC-38 cells was subsequently placed on differentiated adipocytes, and the amount of glycerol released into the medium was quantified. Glycerol release into the medium is a marker for triglyceride lipolysis in adipocytes (27).

SVF preadipocytes were differentiated into adipocytes as follows: On day −3, SVF preadipocytes were set up in 1 ml medium B at a density of 1×10⁶ cells/well of 12-well plates or 3.3×10⁵ cells/well of 48-well plates. On day −2, medium was removed and replaced with fresh 1 ml (12-well format) or 250 μl (48-well format) of medium B. On day 0, medium was removed, and differentiation was initiated with 1 ml (12-well format) or 250 μl (48-well format) of medium B with 0.5 mM IBMX (MilliporeSigma), 1 μM dexamethasone, and 10 μg/ml insulin (Cayman Chemical). On day 2, the medium was removed and replaced with fresh 1 ml (12-well format) or 250 μl (48-well format) of medium B with 10 μg/ml insulin. On day 4 and day 6, the medium was removed and replaced with fresh 1 ml (12-well format) or 250 μl (48-well format) of medium B. On day 7 or 8, medium was removed, and cells were washed twice with 1 ml (12-well format) or 250 μl (48-well format) of PBS, followed by addition of 1.5 ml (12-well format) or 300 μl (48-well format) of medium E with the indicated treatment. After treatment for the indicated time, 20-30 μl of medium from each well containing differentiated SVF adipocytes was aliquoted into 96-well plates (Thermo Fisher Scientific, catalog 12-565-501), followed by addition of 70 μl of PBS and 100 μl of free glycerol reagent. Each 96-well plate also contained a glycerol curve of a known amount of glycerol in 100 μl of PBS. After 2 minutes, the amount of absorbance per well was measured using a Tecan microplate reader (absorbance 540 nm). The amount of glycerol concentration released into the medium per condition over background was calculated using the measured absorbance relative to the absorbance of the standardized curve.

Adipocytes exposed to conditioned medium containing C26c20 tumor secretory factors had about 6-fold more glycerol secreted into the medium compared with adipocytes exposed to conditioned medium from control MC-38 cells (data not shown).

IL-6 and TNFα are secreted factors known to induce lipolysis. To rule out these proteins as lipolytic factors in the medium, we used antibodies directed against IL-6 and TNFα to neutralize their activity. Commercially available recombinant proteins and antibodies were obtained for IL-6 and TNFα. The antibodies against IL-6 and TNFα neutralized the lipolysis activities of their respective recombinant proteins (data not shown). However, these antibodies were unable to neutralize the lipolysis activity induced by medium of C26c20 cells (data not shown).

### Example 2

Identification of LIF as a Secreted Factor from C26c20 Cells that Induces Adipocyte Lipolysis.

To identify the proteins that mediated lipolysis in C26c20 conditioned medium, we carried out a biochemical purification using the lipolysis assay described above using SVF preadipocytes differentiated into adipocytes set up in 12-well plates or 48-well plates. On day 7 or 8, medium was removed, and cells were washed twice with 1 ml (12-well format) or 250 μl (48-well format) of PBS, followed by addition of 1.5 ml (12-well format) or 300 μl (48-well format) of medium with the indicated treatment. After treatment for the indicated time, 20-30 μl of medium from each well containing differentiated SVF adipocytes was aliquoted into 96-well plates (Thermo Fisher Scientific, catalog 12-565-501), followed by addition of 70 μl of PBS and 100 μl of free glycerol reagent. Each 96-well plate also contained a glycerol curve of a known amount of glycerol in 100 μl of PBS. After 2 minutes, the amount of absorbance per well was measured using a Tecan microplate reader (absorbance 540 nm). The amount of glycerol concentration released into the medium per condition over background was calculated using the measured absorbance relative to the absorbance of the standardized curve.

Partial Purification of Lipolysis Activity from C26c20 Conditioned Medium.

The overall scheme of the enrichment of lipolysis activity from the CX tumor-conditioned medium is summarized in Table 1. All operations detailed were carried out on ice or at 4° C., and the assay described above was used to follow triglyceride lipolysis activity through each of the 4 steps outlined in the Table 1 purification. C26c20 medium was prepared as described above. Medium (˜1.5 l) was thawed and dialyzed against 61 of buffer A overnight, followed by centrifugation at 10,000 g for 15 minutes. The supernatant was collected (Table 1, Step 1) and loaded onto a 5-ml SP-Sepharose ion-exchange column (Hi Trap SP HP, pH 7.5, GE Healthcare) preequilibrated in buffer A. The columns were washed with 8 column volumes of buffer A, and bound proteins were eluted with a continuous NaCl gradient (0-1 M) in buffer A over 10 column volumes. Lipolysis active elution fractions were combined (Table 1, Step 2) and concentrated from 40 ml to 1 ml using a 10,000 MWCO Amicon Ultra centrifugal filter that was prewashed in buffer A. The concentrated material was loaded onto gel-filtration chromatography (Tricorn 10/300 Superdex 200 column; GE Healthcare) preequilibrated with buffer B containing 100 mM NaCl (Table 1, Step 3). The peak fractions were combined and diluted with buffer B to dilute NaCl to ˜25 mM. This material was subsequently loaded onto a 1-ml Q-Sepharose ion-exchange column (Hi Trap Q HP, pH 7.5, GE Healthcare) preequilibrated in buffer B. The column was washed with 10 column volumes of buffer B, and bound proteins were eluted with a continuous NaCI gradient (0-1 M). Lipolysis activity was found in the flow-through (Table 1, Step 4).

Lipolysis activity was partially purified >250-fold with a 2% recovery in 2 separate purifications. The partially purified complex fraction from each of the separate purifications was subjected to mass spectrometry analysis, yielding peptides from 118 significant unique proteins that were present in both purifications. Only 26 of these proteins contained a signal sequence, an expected characteristic of secreted factors. Of these signal sequence-containing proteins, LIF was among the top 10 proteins with the highest peptide spectra matches elicited from mass spectrometry analysis (Table 2). We focused on LIF from this list for further studies, owing to its previous association with muscle wasting in cancer CX (18) and body weight changes when expressed in or administered to animals (16, 17). Furthermore, LIF's RNA expression is highest in human cancers (lung, gastrointestinal, and renal) that are associated with cancer CX (data not shown).

TABLE l
Partial Purification of Adipocyte Lipolysis Activity from
Cachexia-inducing Tumor Medium.

| | | | Total | Specific | | |
|---|---|---|---|---|---|---|
| | | Protein^{a} | Activity^{b} | Activity | Purification | Recovery |
| Step | Fraction | (mg) | (fmol) | (fmol/mg) | (fold) | (%) |
| 1 | Cachexia Cell Medium^{c} | 1,249.2 | 94,327 | 76 | — | 100 |
| 2 | SP-Sepharose (pH 7.5) | 2.9 | 15,000 | 5,178 | 69 | 16 |
| 3 | Superdex 200 10/300 | 0.2 | 2,872 | 12,054 | 160 | 3 |
| 4 | Q-Sepharose (pH 7.5) | 0.1 | 2,038 | 19,506 | 258 | 2 |

^{a}Protein concentration of the pooled fractions containing activity was determined as described.
^{b}Adipocyte lipolysis activity was determined as described.
^{c}The starting fraction contains at least two proteins with lipolysis activity LIF (~20% of total activity) and unidentified protein(s) (~80% of total activity).

TABLE 2
Proteins Containing Signal Sequences with Peptides Identified in Purification of
Cachexia-Inducing Tumor Medium.

| | | | | | Length | MW | Pep. | % |
|---|---|---|---|---|---|---|---|---|
| # | PSM^{a} | Accession | Symbol | Protein Description | (AA) | (Da) | Seqs^{b} | Cov.^{c} |
| 1 | 761 | P06797 | CATL1 | Cathepsin L1 | 334 | 37,613 | 34 | 91 |
| 2 | 465 | P12032 | TIMP1 | Metalloproteinase inhibitor 1 | 205 | 22,667 | 19 | 74 |
| 3 | 294 | P14719-2 | ILRL1 | Isoform B of Interleukin-1 receptor-like 1 | 337 | 38,567 | 22 | 64 |
| 4 | 254 | Q01149 | CO1A2 | Collagen alpha-2(I) | 1,372 | 129,875 | 63 | 71 |
| 5 | 156 | P11276 | FINC | Fibronectin | 2,477 | 273,106 | 47 | 25 |
| 6 | 136 | Q8BND5-3 | QSOX1 | Isoform 3 of Sulfhydryl oxidase 1 | 568 | 63,458 | 38 | 71 |
| 7 | 126 | Q61468 | MSLN | Mesothelin | 625 | 69,559 | 20 | 35 |
| 8 | 105 | P20060 | HEXB | Beta-hexosaminidase subunit beta | 536 | 61,221 | 28 | 53 |
| 9 | 104 | P09056 | LIF | Leukemia inhibitory factor | 203 | 22,327 | 15 | 52 |
| 10 | 100 | P47880 | IBP6 | Insulin-like growth factor-binding protein6 | 238 | 25,384 | 19 | 70 |
| 11 | 95 | P28798 | GRN | Granulins | 589 | 65,152 | 28 | 61 |
| 12 | 64 | Q61398 | PCOC1 | Procollagen C-endopeptidase enhancer1 | 468 | 50,262 | 20 | 58 |
| 13 | 49 | Q9R045 | ANGL2 | Angiopoietin-related protein 2 | 493 | 57,214 | 15 | 31 |
| 14 | 44 | Q62181 | SEM3C | Semaphorin-3C | 751 | 85,451 | 18 | 32 |
| 15 | 42 | P21460 | CYTC | Cystatin-C | 140 | 15,557 | 10 | 54 |
| 16 | 27 | Q8QZR4 | OAF | Out at first protein homolog | 282 | 31,576 | 14 | 53 |
| 17 | 16 | P08122 | CO4A2 | Collagen alpha-2(IV) chain | 1,707 | 167,724 | 8 | 9 |
| 18 | 14 | P19324 | SERPH | Serpin H1 | 417 | 46,612 | 10 | 35 |
| 19 | 11 | P47879 | IBP4 | Insulin-like growth factor-binding protein4 | 254 | 27,861 | 7 | 32 |
| 20 | 9 | P08121 | CO3A1 | Collagen alpha-1(III) chain | 1,464 | 139,290 | 5 | 6 |
| 21 | 6 | Q8R0F3 | SUMF1 | Sulfatase-modifying factor 1 | 372 | 40,742 | 3 | 13 |
| 22 | 5 | P06869 | UROK | Urokinase-type plasminogen activator | 433 | 48,363 | 3 | 11 |
| 23 | 5 | P26262 | KLKB1 | Plasma kallikrein | 638 | 71,516 | 2 | 3 |
| 24 | 5 | Q91ZV3 | DCBD2 | Discoidin, CUB and LCCL domain protein 2 | 769 | 83,937 | 3 | 5 |
| 25 | 3 | P11087 | CO1A1 | Collagen alpha-1(I) chain | 1,453 | 118,108 | 2 | 2 |
| 26 | 2 | Q64299 | NOV | Protein NOV homolog | 354 | 38,991 | 3 | 11 |

^{a}Peptide Spectrum Matches (PSM) = Total number of identified peptide spectra of the respective protein.
^{b}Pep. Seqs = Unique identified peptide sequences of respective protein.
^{c}% Cov = Percentage of respective protein sequence matched by identified peptides.

To be considered a relevant driver of cancer CX-induced lipolysis, we would expect LIF expression to be increased primarily in CX-inducing cancer cell lines. Medium from the CX-(C26c2) and non-CX-inducing (MC-38) cell lines, which demonstrated a significant difference in lipolysis activity, were subjected to SDS/PAGE followed by immunoblot (IB) analysis of LI. There was a greater than 10-fold increase in LIF protein in the C26c20 CX medium versus the MC-38 non-CX control medium with similar amounts of total protein found in medium of both cell lines as judged by Ponceau S stain of the membrane (data not shown). When partially purified C26c20 medium was immunodepleted of LIF, lipolysis activity was decreased to background levels (data not shown), consistent with the hypothesis that LIF was responsible for the lipolysis activity in our partially purified material.

To confirm that L induced lipolysis, we produced recombinant murine LIF with an N-terminal His6 tag followed by a TEV protease site in bacteria.

Plasmid construction. All constructs were cloned into the pRSET B expression vector (Thermo Fisher Scientific). pLIF encoding N-terminal His6-tagged followed by a TEV protease cleavage site—and then a signal-peptide-deficient murine LIF (amino acids 24-203)—was purchased from GenScript. This construct is referred to as rLIF. Mutation of lysine to alanine at amino acid 159 of rLIF (rLIF K159A) was produced by site-directed mutagenesis of the above construct using QuikChange II XL kit (Agilent Technologies).

Purification of rLIF from E. coli.

WT and mutant plasmids were transformed into BL21(DE3) pLysS Escherichia coli-competent cells (MilliporeSigma), followed by cell induction with 1 mM IPTG at 18° C. for 16 hours. Hereafter, all operations were carried out at 4° C. unless otherwise stated. A cell pellet from 6 l bacterial culture was resuspended and incubated for 30 minutes in 100 ml of buffer D containing 1 mg/ml lysozyme, 0.4 mg/ml PMSF, and protease inhibitor cocktail (1:1,000). The cells were lysed with a dounce homogenizer, followed by treatment with a tip sonicator with 3 intervals of 3-second pulses over 3 minutes, with 10 minutes of rest on ice. This material was then subjected to centrifugation at 100,000 g for 60 minutes. The resulting supernatant was filtered using a 250 ml, 0.2 μm filter apparatus (MilliporeSigma) and subsequently loaded onto a 1-ml His Trap HP nickel column preequilibrated with buffer C. The column was washed sequentially with 10 column volumes of buffer C, followed by 10 column volumes of buffer C with 10 mM imidazole. Bound protein was eluted in 5-ml fractions with buffer A containing a linear gradient of 10-500 mM imidazole. The eluted fractions were combined and concentrated to 2 ml using a 10,000 MWCO Amicon Ultra centrifugal filter and then subjected to size-exclusion chromatography (Tricorn 10/300 Superdex 200 column; GE Healthcare) preequilibrated with buffer C. Protein-rich fractions were pooled and protein concentrations were quantified using a NanoDrop Instrument (Thermo Fisher Scientific). This material was incubated with purified TEV protease at a ratio of 2 mg purified rLIF to 1 mg purified His-tagged TEV protease in a final concentration of 0.2 mg/ml in buffer A overnight at 4° C. to remove the His6 tag on rLIF. The cleaved material was loaded onto a 1-ml His Trap HP nickel column preequilibrated with buffer C. The column was washed sequentially with 10 column volumes of buffer A, followed by 10 column volumes of buffer C with 10 mM imidazole. Although void of its His6 tag, rLIF still bound the nickel column and was eluted in a 2 ml fraction with buffer C with 50 mM imidazole. The eluted fractions were combined and concentrated to 2 ml using a 10,000 MWCO Amicon Ultra centrifugal filter and then subjected to size-exclusion chromatography (Tri-corn 10/300 Superdex 200 column; GE Healthcare) preequilibrated with endotoxin-free PBS. Protein-rich fractions were pooled and loaded onto a 1.5-ml Polymyxin B column (GenScript; Toxin Eraser Endotoxin Removal Kit) preequilibrated in endotoxin-free PBS, and flow-through was collected. The flow-through was reloaded onto the 1.5-ml Polymyxin B column, and this sequence was repeated a total of 10 times. The final flow-through material was brought up to a final concentration of 1 mg/ml in endotoxin-free PBS. Final purified material endotoxin levels were quantified to ensure that levels were less than 0.05 EU/μg purified LIF (GenScript; ToxinSensor Chromogenic LAL Endotoxin Assay Kit).

WT rLIF was purified using a combination of nickel and size-exclusion chromatography before and after the removal of the N-terminal His6 tag by TEV protease treatment. Buffer (1 ml) containing 5-6 mg of rLIF (red) or rLIF K159A (blue) was loaded on to a Tricorn 10/300 Superdex 200 column and chromatographed at a flow rate of 0.5 ml/min. Absorbance at 280 nm (A280) was monitored continuously to identify rLIF (red) and rLIF K159A (blue). Maximal A280 values for each protein (rLIF: 576 mAU and rLIF K159A: 728 mAU) were normalized to 1. Gel-filtration chromatography (FIG. 1A) of purified rLIF eluted as a single sharp peak at its expected monomer molecular weight (MW) (˜20 kDa), and its homogeneity was confirmed by Coomassie staining (FIG. 1A, inset). 4 μg of each indicated protein was subjected to 15% SDS/PAGE and stained with Coomassie. Endotoxin was removed below 0.05 EU/μg protein from purified recombinant protein by Polymyxin B chromatography.

Previous efforts identified a point mutation (K159A) in LIF that abolishes LIF's interaction with its receptor (28). rLIF K159A has a >100-fold decrease in binding affinity to LIFR-α and activation of the JAK/STAT pathway compared with WT LIF (28); therefore, we compared the activity of LIF and LIF K159A to induce lipolysis. The mutant LIF (rLIF K159A) was purified using the same expression and purification scheme as for WT rLIF. Purified rLIF K159A eluted as a single sharp peak, similar to WT rLIF on gel-filtration chromatography (FIG. 1A), and its homogeneity was confirmed by Coomassie staining (FIG. 1A, inset).

Using the tissue culture adipocyte lipolysis assay, we tested both purified recombinant versions of LIF for their ability to stimulate lipolysis. FIG. 1B-D show adipocyte lipolysis and signaling in wild-type and mutant rLIF-treated adipocytes. Differentiated adipocytes in a 12-well format were treated in a final volume of 1.5 ml of medium supplemented with either the indicated concentration of rLIF or rLIF K159A (FIG. 1B-C); or 30 nM Isoprotererenol or 1 ng/ml rLIF in the absence or presence of the indicated concentration of Atglistatin (FIG. 1D). After incubation for 20 h at 37° C., medium was collected and glycerol concentration was measured using the adipocyte lipolysis assay (FIGS. 1B and D) or adipocyte cells were harvested and subjected to IB analysis (FIG. 1C) (10 g/lane) with the indicated antibody. Each data point (FIG. 1B) represents the mean±SEM of the relative change in medium glycerol concentration compared to conditions containing rLIF without Atglistatin (red, 26 μM) or isoproterenol without Atglistatin (black, 83 μM). As shown in (FIG. 1B), in vitro adipocyte lipolysis activity with WT rLIF was increased over background by ˜5-fold compared with mutant rLIF K159A.

### Example 3

LIF Induces Adipocyte ATGL-Mediated Lipolysis Through GP130 and JAK/STAT Activation.

To further interrogate LIF's mechanism for induction of lipolysis, we tested LIF's capacity to signal through its canonical pathway activated in other tissues. LIF binds to the LIFR-α receptor and gp130 coreceptor, causing activation of the JAK/STAT pathway in multiple tissues, including muscle and endometrium (18, 29).

To confirm that LIF uses its typical coreceptor gp130 to induce lipolysis, we attempted to neutralize LIF-induced lipolysis with antibody against gp130. SVF adipocytes were differentiated as described above in a 48-well format Differentiated adipocytes in 48-well format were treated in a final volume of 300 μl of medium supplemented with 1 ng/ml rLIF in the absence or presence of 3 μg/ml of the indicated antibody. After incubation for 20 h at 37° C., medium was collected and glycerol concentration was measured using the adipocyte lipolysis assay as described above. Data is shown in FIG. 1E as dot plots with bars representing mean±SEM of the relative change in medium glycercol concentration compared to conditions containing rLIF without antibody (32 μM). These results were confirmed in two FIG. 1E or three FIG. 1A-D independent experiments. pSTAT3=phosphorylated STAT3. *p<0.05, *p<0.01, and ***p<0.001 based on Student's t-test.

As shown in FIG. 1E, LIF's ability to induce lipolysis was blocked with antibodies to LIF itself or to the gp130 coreceptor, but not with IL-6 antibody. Further downstream, we interrogated JAK/STAT activation. The adipocytes described in FIG. 1B were processed for IB analysis. As shown in FIG. 1C, treatment of adipocytes with increasing concentrations of WT rLIF (lanes 2-4) showed a corresponding increase in the phosphorylation of STAT1 (third panel) and STAT3 (first panel), while rLIF K159A (lanes 5-7) had no effect on the phosphorylation of these proteins. Total STAT1 (fourth panel) and STAT3 (second panel) protein levels remained constant among the groups.

In adipocytes, triglycerides are sequentially hydrolyzed by ATGL, HSL, and monoacylglycerol lipase (MGL). Data from others suggested that CX-induced lipolysis requires ATGL activity (8). To determine if ATGL is involved in LF-induced lipolysis, we incubated adipocytes with rLIF or isoproterenol in the absence or presence of increasing concentrations of the ATGL inhibitor Atglistatin. As shown in FIG. 1D, Atglistatin treatment of adipocytes only inhibited rLIF-induced lipolysis but not lipolysis induced by isoproterenol.

### Example 4

LIF Induces Loss of Adipose Tissue and Body Weight without Persistent Alteration in Food Intake.

To determine whether LIF induces lipolysis and a CX-like phenotype in vivo, we administered rLIF to mice syngeneic to the C26c20 tumor line, Balb/c. Chow-fed Balb/c mice (10-week-old males) were housed four mice per cage and injected i.p. with 100 μl PBS in the absence or presence of recombinant Leukemia Inhibitory Factor (rLIF) or rLIF K159A at 80 μg/kg body weight twice daily throughout the experiment. Mice were then monitored for changes in body weight and food intake, as well as ECHO MRI changes of fat and lean body mass. Body weight FIG. 2A, food intake FIG. 2B, and ECHO MRI measurements of fat mass FIG. 2C were measured at 9 a.m. at the indicated time points. Body weight and fat mass are shown relative to the average day 0 reference value for each respective cohort.

As shown in FIG. 2A, mice injected with rLIF lost ˜15% of their body weight compared with mice treated with either PBS or rLIF K159A. The average values for body weight FIG. 2A at day 0 were 21.3, 21.8 and 22.3 g for the PBS, rLIF, and rLIF K159A-treated mice, respectively. There was a reduction in food intake in the first 6 days of the experiment in rLIF-administered mice compared with mice injected with PBS or rLIF K159A (FIG. 2B, left panel). However, this difference resolved over days 7-21, and there was no overall significant difference in daily food intake over the 21 days of the experiment (FIG. 2B, right panel).

ECHO MRI results revealed that mice injected with rLIF lost more than 50% of their adipocyte mass, whereas mice receiving PBS or rLIF K159A injections had no significant change in fat mass (FIG. 2C). The average values for fat mass FIG. 2C at day 0 were 2.4, 3.1 and 3.1 g for the PBS, rLIF, and rLIF K159A-treated mice, respectively. Each value represents mean±SEM of four mice. Lean mass was minimally changed in rLIF-administered mice (data not shown).

At the conclusion of the experiment, epididymal white adipose tissue (eWAT), gastrocnemius muscle, liver, and spleen were harvested, fixed in formalin, and stained with H&E (FIG. 2D). Epididymal white adipose tissue (eWAT), gastrocnemius muscle, liver and spleen were harvested 21 days after start of injections. Representative sections stained with hematoxylin and eosin FIG. 2D of each of these tissues are shown. Magnification, 40×; scale bar, 270 μm. Additionally, eWAT from two representative mice from each cohort was processed, and aliquots of cell lysate (15 μg/lane) were subjected to IB analysis FIG. 2E with the indicated antibodies. Histology of the eWAT (FIG. 2D) showed significant atrophy of adipocytes from mice administered rLIF (middle panel) compared with mice receiving PBS (top panel) or rLIF K159A (bottom panel). There were limited differences in the histology of muscle, liver, or spleen among cohorts (data not shown). eWAT was also processed for IB analysis. As shown in FIG. 2E, adipose tissue from mice that received rLIF (lanes 3-4) had increased phosphorylated STAT1 (pSTAT1; middle panel) and pSTAT3 (top panel), with no detectable STAT phosphorylation in adipose tissue from mice treated with PBS (lanes 1-2) or rLIF K159A (lanes 5-6). This finding parallels the in vitro rLIF activation of STAT signaling in adipocytes (FIG. 1C).

These results were confirmed in at least three independent experiments. pSTAT1=phosphorylated STAT1; pSTAT3=phosphorylated STAT3. *p<0.05 and **p<0.01 based on Student's t-test FIG. 2B or p-value based on use of Generalized Estimated Equation approach comparing multiple groups over time with rLIF-treated mice as the reference value FIGS. 2A and C.

### Example 5

Decreased Leptin Signaling Overcomes LIF's Anorexic Effects.

rLIF administration to WT mice induced an early transient state of hypophagia that returned to normophagia over 6 days. We hypothesized that there may be an interplay between LIF administration and leptin levels affecting overall net food intake.

Mouse models that lack leptin are in the C57BL/6J background; therefore, we first confirmed that rLIF administered to WT C57BL/6J mice also induces adipose loss without persistent hypophagia. Chow-fed C57BL/6 mice (9-week-old males) were housed four mice per cage and injected i.p. with 100 μl PBS in the absence or presence of rLIF or rLIF K159A at 80 μg/kg body weight twice daily throughout the experiment. ECHO MRI measurements of fat mass and lean mass, and food intake, were measured at 9 a.m. at the indicated time points. Fat mass and lean mass are shown relative to the average day 0 reference value for each respective cohort.

The average values for fat mass at day 0 were 2.8, 2.9 and 2.8 g for the PBS, rLIF, and rLIF K159A treated mice, respectively. The average values for lean mass at day 0 were 16.5, 16.8 and 17.0 g for the PBS, rLIF, and rLIF K159A-treated mice, respectively. Each value represents mean±SEM of eight mice.*p<0.05 based on Student's t-test or p-value based on use of Generalized Estimated Equation approach comparing multiple groups over time with rLIF-treated mice as the reference value. N.S.=not significant.

WT C57BL/6J mice administered rLIF lost ˜40% of their adipose mass compared with mice receiving PBS or rLIF K159A (data not shown). There were no significant changes in lean muscle mass (data not shown). rLIF administration reduced food intake during the first 6 days compared with mice administered PBS or rLIF K159A (data not shown). The timing of the acute decrease in food intake was associated with the period of maximal fat loss in the rLIF-treated mice (data not shown). This transient hypophagia reversed, and food intake returned to baseline levels during days 7-24 (data not shown).

To evaluate the transition of hypophagia to normophagia in rLIF-administered WT mice, we conducted an experiment to determine the change in serum leptin concentrations as a function of adipose loss and food intake. Chow-fed C57BL/6 mice (8-week-old males) were housed four mice per cage and injected i.p. with 100 μl PBS in the absence or presence of rLIF at 80 μg/kg body weight twice daily throughout the experiment. ECHO MRI measurements of fat mass FIG. 3A, food intake FIG. 3C, and serum leptin FIG. 3B was measured and are shown relative to the PBS-treated control mice at the indicated time point.

Again, we observed a decrease in fat mass (FIG. 3A), with the majority of the fat loss occurring between days 1 and 8 in rLIF-administered mice compared with PBS control. The average values for fat mass (FIG. 3A) of PBS-treated control mice were 2.8, 2.7, 2.8, 3.0, 3.0 and 3.2 g for days 0, 4, 8, 12, 16 and 20, respectively. The average values for leptin concentration (FIG. 3B) of PBS-treated control mice were 3.0, 2.0, 2.9, 2.7, 3.2 and 3.3 ng/ml for days 0, 4, 8, 12, 16 and 20, respectively. Each value represents mean±SEM of eight mice. The timing of adipose tissue loss in rLIF-treated mice coincided with an interval of acute hypophagia (FIG. 3C, days 2-7). Leptin levels decreased between days 1 and 8, in association with the loss of adipose tissue (FIG. 3B). It is during this time that the acute hypophagia observed with initial rLIF treatment also resolved, with a return to normophagia (FIG. 3C), compare days 2-7 vs. days 8-14 and 15-20).

Leptin signals proopiomelanocortin (POMC) neurons of the hypothalamus through the STAT3 pathway to regulate appetite (30, 31). LIFR-α is also expressed in POMC neurons of the hypothalamus, and central intracerebroventricular administration of LIF to WT mice increased STAT3 phosphorylation in these neurons, which induced an immediate, limited (4-hour) anorexic effect (32).

We measured hypothalamic STAT3 phosphorylation by IB analysis of rLIF-injected WT C57BL/6J mice to evaluate if peripheral administration of rLIF increased STAT3 phosphorylation. Chow-fed C57BL/6 mice (11-week-old males) were housed three mice per cage and injected with PBS in the absence or presence of rLIF at 80 μg/kg body weight i.p. twice daily for the indicated time frame and food intake was measured. Every three days, three mice from each cohort were sacrificed followed by harvesting and processing of the hypothalamus. Aliquots (30 μg/lane) of pooled hypothalamic cell lysate (FIG. 3D) or individual mouse hypothalamic cell lysate (FIG. 3E) from three mice treated identically were subjected to IB analysis with the indicated antibodies. The unfilled circles represent the average food intake from the day the three mice from each cohort were sacrificed for hypothalamic processing. As shown in FIG. 3D, hypothalamic STAT3 phosphorylation increased over 6 days in rLIF-injected mice (IB, top panel, lanes 6-8) compared with PBS-treated animals (IB, top panel, lanes 1-3). During this time, there was an associated decrease in food intake in rLIF-administered mice (FIG. 3D, graph, red, days 1-3 and 4-6). As shown in FIG. 3D, as the food intake returned to normal levels (graph, red, days 7-9 and 10-12), there was also a decrease in measured hypothalamic STAT3 phosphorylation (IB, top panel, lanes 9-10) down to pretreatment levels (IB, top panel, compare lanes 6 and 10). FIG. 3E details the hypothalamic STAT3 phosphorylation of each individual rLIF-treated mouse of the experiment in FIG. 3D. Together, the experiments of FIG. 3 suggest that peripheral administration of rLIF increases the phosphorylation of hypothalamic STAT3, which is associated with acute hypophagia. As adipose tissue and leptin levels decrease, there is an associated decrease in hypothalamic STAT3 phosphorylation back to pretreatment levels that corresponds with a return to normophagia. FIG. 3A-E These results were confirmed in three independent experiments. *p<0.05, **p<0.01, and ***p<0.001 based on Student's t-test comparing rLIF-treated mice to PBS-treated mice at the indicated time FIG. 3A-D. pSTAT3=phosphorylated STAT3.

To validate that decreased leptin levels in rLIF-treated WT mice were responsible for a return of food intake from hypophagia to normophagia, we coadministered rleptin to our rLIF-injected mouse model. Mice received rLIF for 15 days, resulting in a loss of adipose mass of ˜30%-40% and a body weight loss of ˜10%. On day −15, chow-fed C57BL/6 mice (11-week-old males) were injected i.p. with 100 μl PBS containing 80 μg/kg body weight rLIF twice daily for 15 days with average fat mass loss of ˜30-40% and weight loss ˜10%. Mice were then randomized into 4 groups receiving injections containing PBS, rLIF, rleptin, or a combination of rLIF and rleptin. On day 0, mice were randomized and housed four mice per cage and treated with 100 μl PBS in the absence or presence of 80 μg/kg body weight rLIF twice daily and/or 5 mg/kg leptin once daily for 9 days. ECHO MRI measurements of fat mass (FIG. 4A), body weight (FIG. 4B), and food intake (FIG. 4C) were measured at the indicated time points and are shown relative to the average day 0 reference value for each respective cohort. The average day 0 values for fat mass were 1.7, 1.7, 1.6, and 1.7 g and body weight were 22.1, 23.5, 21.5 and 23.1 g for the PBS, rLIF, leptin, and rLIF plus leptin cohorts, respectively. Each value represents dot plot with mean±SEM FIG. 4A or mean±SEM (FIG. 4B-C) of four mice. These results were confirmed in two independent experiments. *p<0.05, **p<0.01, and ***p<0.001 based on Student's t-test FIG. 4C or p-value based on use of Generalized Estimated Equation approach comparing multiple groups over time with rLIF+rleptin-treated mice as the reference value FIG. 4A-B.

As shown in FIG. 4A, mice receiving the combination of rLIF and reptin had continued hypophagia compared with mice injected with PBS and rLIF. The coadministered rLIF and rleptin mice also had a further decrease in fat mass (FIG. 4B) and body weight (FIG. 4C) compared with mice injected with PBS, rLIF, and rleptin. These results suggest that giving leptin back to rLIF-chronically treated mice reversed the counterbalance of reduced leptin levels, allowing rLIF's central effect of hypophagia to persist and resulting in further body weight and adipose loss.

### Example 6

LIF Induces Loss of Adipose Tissue in Ob/Ob Mice.

Next, we determined if rLIF induced weight loss in mouse models of obesity that lack leptin signaling. Eleven-week-old male ob/ob mice were injected with either PBS, rLIF, or rLIF K159A (80 Kg/kg i.p.) twice daily for 48 days (treatment phase), followed by another 29 days without injections (posttreatment phase). Mice were monitored for changes in body weight, food intake, and ECHO MRI changes of fat and lean mass levels throughout the experiment (FIG. 5).

Chow-fed Lep^{ob}/J, ob/ob, mice (10-week-old males) were housed four mice per cage and injected i.p. with 100 μl PBS in the absence or presence of rLIF or rLIF K159A at 80 μg/kg body weight twice daily for 48 days (treatment) and subsequently followed for another 29 days (post-treatment) without injections. Body weight (FIG. 5A), food intake (FIG. 5C), and ECHO MRI measurements of fat mass (FIG. 5B) were measured at the indicated time points for 77 days. Body weight and fat mass are shown relative to the average day 0 reference value for each respective cohort. The average values for body weight (FIG. 5A) at day 0 were 39.5, 39.5 and 39.3 g for the PBS, rLIF, and rLIF K159A treated mice, respectively. The average values for fat mass FIG. 5B at day 0 were 20.1, 20.6 and 20.9 g for the PBS, rLIF, and rLIF K159A-treated mice, respectively.

As shown in FIG. 5A, mice injected with rLIF during the treatment phase (left panel) had a >30% decrease in body weight compared with mice treated with either PBS or rLIF K159A. Once injections were discontinued (right panel), the rLIF-injected cohort rapidly gained body weight, approaching the body weights measured in mice treated with PBS or rLIF K159A. The decrease in body weight found during the treatment phase in ob/ob mice receiving rLIF correlated with the decrease in adipose tissue mass quantified by ECHO MRI (FIG. 5B, left panel). These mice also demonstrated a steep increase in their fat mass during the posttreatment phase, matching that found in the control cohorts (FIG. 5B, right panel). No differences were observed in lean tissue mass during the treatment and posttreatment phases (data not shown). Food intake of ob/ob mice treated with rLIF was reduced by ˜50% compared with mice injected with either PBS or rLIF K159A (FIG. 5C, left panel). The change in food intake remained persistent during the entire treatment phase, resulting in a significant difference in food intake in mice receiving rLIF (data not shown). This is in contrast to the findings shown in FIG. 2B and FIG. 3C, in which rLIF administration to WT mice with an intact leptin system did not persistently alter food intake.

We next measured hypothalamic STAT3 phosphorylation by IB analysis of rLIF-injected ob/ob mice to evaluate if peripheral administration of rLIF increased STAT3 phosphorylation. Chow-fed Lep^{ob}/J, ob/ob, mice (10-week-old males) were housed three mice per cage and treated with PBS or rLIF as above for the indicated time interval and food intake was measured. Every three days, three mice from each cohort were sacrificed followed by harvesting and processing of the hypothalamus as described in the Methods. Aliquots (30 μg/lane) of pooled hypothalamic cell lysate from three mice treated identically were subjected to IB analysis with the indicated antibodies as described. The unfilled circles in FIG. 5 (A-D) represent the average food intake from the day the three mice from each cohort were sacrificed for hypothalamic processing. Each value represents mean±SEM (FIG. 5A-C) or dot plots with mean±SEM (FIG. 5D-F) of three mice (FIG. 5D) or four mice (FIG. 5A-C). These results were confirmed in two (FIG. 5D) or three independent experiments (FIG. 5A-C). *p<0.05, **p<0.01, and ***p<0.001 based on Student's t-test comparing rLIF-treated mice to PBS or rLIF K159A-treated mice over the respective time interval FIG. 5C-D or p-value based on use of Generalized Estimated Equation approach with rLIF-treated mice as the reference value FIG. 5A-B. pSTAT3=phosphorylated STAT3 and N.S.=not significant.

As shown in FIG. 5D, hypothalamic STAT3 phosphorylation increased and persisted throughout the 15 days of the experiment (IB, top panel, lanes 7-12) compared with PBS-treated animals (IB, top panel, lanes 1-6). There was also a persistent decrease in food intake in rLIF-administered mice (FIG. 5D, graph, red) compared with PBS-treated animals (FIG. 5D graph, black). This data suggest that rLIF-induced hypothalamic STAT3 phosphorylation and hypophagia cannot return toward normal levels in mice that lack the leptin signaling axis. This is in contrast to the finding shown in FIG. 3D, in which rLIF administration to WT mice with an intact leptin system did not maintain a persistent increase in their STAT3 phosphorylation or a persistent decrease in food intake.

### Example 7

LIF has Central and Noncentral Roles in Leptin Signaling-Deficient Mice.

As shown above, rLIF administration persistently decreased food intake in ob/ob mice, which was not observed in WT mice. However, both mouse models displayed a significant loss of adipose tissue and body weight relative to controls, suggesting that, in WT mice, there is an appetite-independent contribution to rLIF's ability to induce adipose loss. To determine if the reduction in food intake in the ob/ob mice was solely responsible for the weight loss, we performed a pair-feeding experiment in which PBS- and rLIF K159A-treated ob/ob animals were restricted to the same daily food intake as rLIF-treated ob/ob animals.

Chow-fed Lep^{ob}/J, ob/ob, mice (11-week-old males) were housed individually and injected i.p. with 100 μl PBS in the absence or presence of rLIF or rLIF K159A at 80 μg/kg body weight twice daily for 18 days throughout the experiment. PBS and rLIF K159A-treated mice were either fed ad libitum or pair fed to the food intake of rLIF-treated mice fed ad libitum. Food intake (FIG. 6A), body weight (FIG. 6B) and ECHO MRI measurement of fat mass (FIG. 6C) were measured at the indicated time points. Body weight and fat mass are shown relative to the average day 0 reference value for each respective cohort. The average values for body weight (FIG. 6B) at day 0 were 44.5, 47.0 and 46.3 g for the PBS, rLIF, and rLIF K159A-treated mice fed ad libitum, respectively. The average day 0 values of body weight for the PBS and rLIF K159A-treated mice pair fed was 45.9 and 46.0 g, respectively. The average values for fat mass (FIG. 6C) at day 0 were 25.4, 24.0 and 24.0 g for the PBS, rLIF, and rLIF K159A-treated mice fed ad libitum, respectively. The average day 0 values of fat mass (FIG. 6C) for the PBS and rLIF K159A-treated mice pair fed were 24.5 and 25.6 g, respectively. Each value represents mean±SEM of four mice. These results were confirmed in two independent experiments. ***p<0.001 based on Student's t-test or p-value based on use of Generalized Estimated Equation approach comparing multiple groups over time with rLIF-treated mice as the reference value FIG. 6A-C.

As shown in FIG. 6A, all the pair-fed PBS—and rLIF K159A—treated mice had the same food intake over 18 days of the experiment as the rLIF-treated mice fed ad libitum. Despite the same food intake, ob/ob mice treated with rLIF lost more weight than those mice fed ad libitum or pair-fed receiving PBS or rLIF K159A (FIG. 6B). The body weight difference corresponded to a 15%-20% decrease in fat mass (FIG. 6C).

LIF promotes decreased food intake and adipose tissue loss in leptin deficiency (ob/ob). Interestingly, leptin has a long-chain helical structure similar to LIF (33), and both induce STAT3 activation. Therefore, we next determined if LIF works directly through the leptin receptor to facilitate weight loss using mice that lack the leptin receptor (db/db) (34). These db/db mice were injected with either PBS, rLIF (80 μg/kg i.p.) or rLIF K159A (80 μg/kg i.p.) twice daily for 47 days (treatment phase), followed by 31 days without injections (posttreatment phase). Mice were monitored for changes in body weight, food intake, and ECHO MRI changes of fat and lean mass throughout the experiment.

Chow-fed Lepr^{db}/J, db/db, mice (8-week-old males) were housed four mice per cage and injected i.p. with 100 μl PBS in the absence or presence of rLIF or rLIF K159A at 80 μg/kg body weight twice daily for 47 days (treatment) and subsequently followed for another 31 days (post-treatment) without injections. Body weight (FIG. 7A), food intake (FIG. 7B), and ECHO MRI measurements of fat mass (FIG. 7C) were measured at 9 a.m. at the indicated time points for 78 days. Body weight and fat mass are shown relative to the average day 0 reference value for each respective cohort. The average values for body weight (FIG. 7A) at day 0 were 35.8, 34.8 and 34.3 g for the PBS, rLIF, and rLIF K159-treated mice, respectively. The average values for fat mass (FIG. 7C) at day 0 were 16.8, 16.7 and 16.9 g for the PBS, rLIF, and rLIF K159A-treated mice, respectively. Each value represents mean±SEM of four mice. These results were confirmed in two independent experiments.***p<0.001 based on Student's t-test (FIG. 7B) or p-value based on use of Generalized Estimated Equation approach comparing multiple groups over time with rLIF-treated mice as the reference value (FIGS. 7A and C). As shown in FIG. 7A, mice injected with rLIF during the treatment phase (left panel) lost ˜30% body weight relative to those mice treated with either PBS or rLIF K159A. Once injections were discontinued (right panel), the rLIF-injected cohort rapidly gained body weight, approaching the body weight observed in mice treated with PBS or rLIF K159A. These results are similar to those of rLIF-injected ob/ob mice (FIG. 5A).

The decrease in body weight measured in db % db mice receiving rLIF during the treatment phase was a result of a decrease in adipose tissue, as quantified by ECHO MRI (FIG. 7C, left panel). Similar to results reported in ob/ob mice, db/db mice had a rapid increase in their fat mass during the posttreatment phase (FIG. 7C, right panel). Limited differences were observed in lean tissue mass among these cohorts during the treatment and posttreatment phases (data not shown). As shown in FIG. 7B (left panel), db/db mice treated with rLIF also showed a persistent decrease in food intake compared with mice injected with either PBS or rLIF K159A during the treatment phase. This finding is similar to rLIF-treated ob/ob mice and again is in contrast to the results obtained in WT mice, in which rLIF administration only transiently decreased food intake (FIG. 2B and FIG. 3C). Combined, our results suggest that rLIF's effects on adipose loss and food intake does not require signaling through the leptin receptor. These results also are consistent with leptin's role to return food intake back to pretreatment levels in rLIF-administered mice.

### Example 8

LIF (rLIF) Acts on Mouse Models of Diet-Induced Obesity to Reduce Weight, Fat Mass, and Food Intake.

FIG. 8 shows that recombinant LIF (rLIF) acts on mouse models of diet-induced obesity to reduce weight, fat mass, and food intake. The rLIF (red) caused a significant decrease in fat mass (FIG. 8A) and body weight (FIG. 8B) when administered to mice compared to mice receiving only vehicle (black). Furthermore, the rLIF reduced food intake in the animals (FIG. 8C). Importantly, by itself, rLIF could only bring fat mass and body weight down only a certain percentage. With co-administration of rLeptin, the present inventors would demonstrate an even more significant and profound weight loss, fat mass loss, and maintenance of decreased food intake.

### REFERENCES

1. Solheim T S, Blum D, Fayers P M, Hjermstad M J, Stene G B, Strasser F, et al. Weight loss, appetite loss and food intake in cancer patients with cancer cachexia: three peas in a pod?—analysis from a multicenter cross sectional study. Acta oncologica (Stockholm, Sweden). 2014; 53(4):539-46.
2. Tisdale M J. Cachexia in cancer patients. Nature reviews Cancer. 2002; 2(11):862-71.
3. Evans W J, Morley J E, Argiles J, Bales C, Baracos V, Guttridge D, et al. Cachexia: A new definition. Clinical nutrition (Edinburgh, Scotland). 2008; 27(6):793-9.
4. Anderson L J, Albrecht E D, and Garcia J M. Update on management of cancer-related cachexia. Current oncology reports. 2017; 19(1):3.
5. Zhou X, Wang J L, Lu J, Song Y, Kwak K S, Jiao Q, et al. Reversal of cancer cachexia and muscle wasting by ActRIIB antagonism leads to prolonged survival. Cell. 2010; 142(4):531-43.
6. Agustsson T, Ryden M, Hoffstedt J, van Harmelen V, Dicker A, Laurencikiene J, et al. Mechanism of increased lipolysis in cancer cachexia. Cancer research. 2007; 67(11):5531-7.
7. Ryden M, Agustsson T, Laurencikiene J, Britton T, Sjolin E, Isaksson B, et al. Lipolysis-not inflammation, cell death, or lipogenesis—is involved in adipose tissue loss in cancer cachexia. Cancer. 2008; 113(7):1695-704.
8. Das S K, Eder S, Schauer S, Diwoky C, Temmel H, Guertl B, et al. Adipose triglyceride lipase contributes to cancer-associated cachexia. Science (New York, N.Y.). 2011; 333(6039):233-8.
9. Bischoff S C, Boirie Y, Cederholm T, Chourdakis M, Cuerda C, Delzenne N M, et al. Towards a multidisciplinary approach to understand and manage obesity and related diseases. Clinical nutrition (Edinburgh, Scotland). 2016.
10. Alamuddin N, Bakizada Z, and Wadden T A. Management of obesity. Journal of clinical oncology: official journal of the American Society of Clinical Oncology. 2016; 34(35):4295-305.
11. Zimmers T A, Fishel M L, and Bonetto A. STAT3 in the systemic inflammation of cancer cachexia. Seminars in cell & developmental biology. 2016; 54:28-41.
12. Song H, and Lim H. Evidence for heterodimeric association of leukemia inhibitory factor (LIF) receptor and gp130 in the mouse uterus for LIF signaling during blastocyst implantation. Reproduction (Cambridge, England). 2006; 131(2):341-9.
13. Auernhammer C J, and Melmed S. Leukemia-inhibitory factor-neuroimmune modulator of endocrine function. Endocrine reviews. 2000; 21(3):313-45.
14. Stewart C L, Kaspar P, Brunet L J, Bhatt H, Gadi I, Kontgen F, et al. Blastocyst implantation depends on maternal expression of leukaemia inhibitory factor. Nature. 1992; 359(6390):76-9.
15. Hu W, Feng Z, Teresky A K, and Levine A J. p53 regulates maternal reproduction through LIF. Nature. 2007; 450(7170):721-4.
16. Akiyama Y, Kajimura N, Matsuzaki J, Kikuchi Y, Imai N, Tanigawa M, et al. In vivo effect of recombinant human leukemia inhibitory factor in primates. Japanese journal of cancer research: Gann. 1997; 88(6):578-83.
17. Beretta E, Dhillon H, Kalra P S, and Kalra S P. Central LIF gene therapy suppresses food intake, body weight, serum leptin and insulin for extended periods. Peptides. 2002; 23(5):975-84.
18. Seto D N, Kandarian S C, and Jackman R W. A key role for leukemia inhibitory factor in C26 cancer cachexia. The Journal of biological chemistry. 2015; 290(32):19976-86.
19. Xu B, Dube M G, Kalra P S, Farmerie W G, Kaibara A, Moldawer L L, et al. Anorectic effects of the cytokine, ciliary neurotropic factor, are mediated by hypothalamic neuropeptide Y: comparison with leptin. Endocrinology. 1998; 139(2):466-73.
20. Gloaguen I, Costa P, Demartis A, Lazzaro D, Di Marco A, Graziani R, et al. Ciliary neurotrophic factor corrects obesity and diabetes associated with leptin deficiency and resistance. Proceedings of the National Academy of Sciences of the United States of America. 1997; 94(12):6456-61.
21. Ettinger M P, Littlejohn T W, Schwartz S L, Weiss S R, McIlwain H H, Heymsfield S B, et al. Recombinant variant of ciliary neurotrophic factor for weight loss in obese adults: a randomized, dose-ranging study. Jama. 2003; 289(14):1826-32.
22. Miller R G, Petajan J H, Bryan W W, Armon C, Barohn R J, Goodpasture J C, et al. A placebo-controlled trial of recombinant human ciliary neurotrophic (rhCNTF) factor in amyotrophic lateral sclerosis. rhCNTF ALS Study Group. Annals of neurology. 1996; 39(2):256-60.
23. Brattain M G, Strobel-Stevens J, Fine D, Webb M, and Sarrif A M. Establishment of mouse colonic carcinoma cell lines with different metastatic properties. Cancer research. 1980; 40(7):2142-6.
24. Soda K, Kawakami M, Kashii A, and Miyata M. Characterization of mice bearing subclones of colon 26 adenocarcinoma disqualifies interleukin-6 as the sole inducer of cachexia. Japanese journal of cancer research: Gann. 1994; 85(11):1124-30.
25. Fox K M, Brooks J M, Gandra S R, Markus R, and Chiou C F. Estimation of cachexia among cancer patients based on four definitions. Journal of oncology. 2009; 2009:693458.
26. Corbett T H, Griswold D P, Jr., Roberts B J, Peckham J C, and Schabel F M, Jr. Tumor induction relationships in development of transplantable cancers of the colon in mice for chemotherapy assays, with a note on carcinogen structure. Cancer research. 1975; 35(9):2434-9.
27. Vaughan M, and Steinberg D. Effect of hormones on lipolysis and esterification of free fatty acids during incubation of adipose tissue in vitro. Journal of lipid research. 1963; 4:193-9.
28. Hudson K R, Vernallis A B, and Heath J K. Characterization of the receptor binding sites of human leukemia inhibitory factor and creation of antagonists. The Journal of biological chemistry. 1996; 271(20):11971-8.
29. Cheng J G, Chen J R, Hernandez L, Alvord W G, and Stewart C L. Dual control of LIF expression and LIF receptor function regulate Stat3 activation at the onset of uterine receptivity and embryo implantation. Proceedings of the National Academy of Sciences of the United States of America. 2001; 98(15):8680-5.
30. Elias C F, Aschkenasi C, Lee C, Kelly J, Ahima R S, Bjorbaek C, et al. Leptin differentially regulates NPY and POMC neurons projecting to the lateral hypothalamic area. Neuron. 1999; 23(4):775-86.
31. Bates S H, Stearns W H, Dundon T A, Schubert M, Tso A W, Wang Y, et al. STAT3 signalling is required for leptin regulation of energy balance but not reproduction. Nature. 2003; 421(6925):856-9.
32. Grossberg A J, Scarlett J M, Zhu X, Bowe D D, Batra A K, Braun T P, et al. Arcuate nucleus proopiomelanocortin neurons mediate the acute anorectic actions of leukemia inhibitory factor via gp130. Endocrinology. 2010; 151(2):606-16.
33. Zhang F, Basinski M B, Beals J M, Briggs S L, Churgay L M, Clawson D K, et al. Crystal structure of the obese protein leptin-E100. Nature. 1997; 387(6629):206-9.
34. Bahary N, Leibel R L, Joseph L, and Friedman J M. Molecular mapping of the mouse db mutation. Proceedings of the National Academy of Sciences of the United States of America. 1990; 87(21):8642-6.
35. Timper K, Denson J L, Steculorum S M, Heilinger C, Engstrom-Ruud L, Wunderlich C M, et al. IL-6 improves energy and hlucose homeostasis in obesity via enhanced central IL-6 trans-signaling. Cell reports. 2017; 19(2):267-80.
36. Komori T, Tanaka M, Senba E, Miyajima A, and Morikawa Y. Deficiency of oncostatin M receptor beta (OSMRbeta) exacerbates high-fat diet-induced obesity and related metabolic disorders in mice. The Journal of biological chemistry. 2014; 289(20):13821-37.
37. Caron A, Labbe S M, Mouchiroud M, Huard R, Lanfray D, Richard D, et al. DEPTOR in POMC neurons affects liver metabolism but is dispensable for the regulation of energy balance. American journal of physiology Regulatory, integrative and comparative physiology. 2016; 310(11):R1322-31.

1. A pharmaceutical composition comprising a first agent in an amount effective for causing both a peripheral effect and a central effect on weight loss, a second agent in an amount effective for causing at least a central effect on weight loss, and one or more pharmaceutically acceptable excipients, wherein the first agent and the second agent are different.

2. The composition according to claim 1, wherein the first agent comprises a member of IL-6 cytokine family or leukemia inhibitory factor.

3. (canceled)

4. The composition according to claim 1, wherein the second agent comprises leptin.

5. A method of causing weight loss comprising administering to a subject a first agent in an amount effective for causing both a peripheral effect and a central effect on weight loss and a second agent in an amount effective for causing at least a central effect on weight loss, wherein the first agent and the second agent are different.

6. The method according to claim 5, wherein the first agent comprises a member of IL-6 cytokine family or leukemia inhibitory factor.

7. (canceled)

8. The method according to claim 5, wherein the second agent comprises leptin.

9. The method of claim 8, wherein the effective amount of leptin administered to the subject is sufficient to counteract a decrease in natural leptin secretion associated with administration of the first agent to the subject.

10. The method according to claim 5, wherein each of the first agent and the second agent is administered to the subject over a period of at least a week, at least a month, at least four months, or at least one year.

11. (canceled)

12. (canceled)

13. (canceled)

14. The method according to claim 5, wherein at least one of the first agent and second agent is administered to the subject by at least one route selected from the group consisting of oral, transmucosal, topical, transdermal, intradermal, subcutaneous, inhalational, intrabronchial, pulmonary, intravenous, intraduodenal, intramuscular and intragastrical.

15. (canceled)

16. The method of claim 5, wherein the subject suffers from obesity, metabolic syndrome, nonalcoholic fatty liver disease, or diabetes mellitus.

17. (canceled)

18. (canceled)

19. (canceled)

20. A method of causing weight loss comprising administering to a subject peripherally a composition comprising an effective amount of an agent that has both a peripheral effect and a central effect on weight loss.

21. The method according to claim 20, wherein the peripheral effect on weight loss comprises lipolysis.

22. The method according to claim 21, wherein the lipolysis occurs in adipocytes.

23. The method according to claim 20, wherein the composition comprises a member of IL-6 cytokine family or leukemia inhibitory factor.

24. (canceled)

25. The method according to claim 20, wherein the composition is administered to the subject enterally or parenterally.

26. The method according to claim 20, wherein the composition is administered to the subject by at least one route selected from the group consisting of oral, transmucosal, topical, transdermal, intradermal, subcutaneous, inhalational, intrabronchial, pulmonary, intravenous, intraduodenal, intramuscular and intragastrical.

27. (canceled)

28. The method according to claim 20, further comprising administering to the subject an effective amount of an additional agent that has a central effect on weight loss.

29. A method of preventing or treating cachexia syndrome comprising administering to a subject in need thereof an effective amount of an agent that inhibits adipose loss.

30. The method according to claim 29, wherein the agent is a monoclonal antibody against leukemia inhibitory factor.

31. The method according to claim 29, wherein the agent reduces gene expression level of leukemia inhibitory factor or blocks leukemia inhibitory factor secretion or signaling.
